SEQUENCE LISTING (6 sequences)

SEQ ID NO: 1 - DNA, 7407 bp, Artificial
  primer
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttggtaccgagctcggatccactagtccagtgtggtggaattctgcagatatccagcacagtggcggccgctcgagggccctgccctgaaggctggtcacttgcagaggtaaactcccctctttgacttctggccagggtttgtgctgagctggctgcagccgctctcagcctcgctccgggcacgtcgggcagcctcgggccctcctgcctgcaggatcatgcccaccaccgtggacgatgtcctggagcatggaggggagtttcactttttccagaagcaaatgtttttcctcttggctctgctctcggctaccttcgcgcccatctacgtgggcatcgtcttcctgggcttcacccctgaccaccgctgccggagccccggagtggccgagctgagtctgcgctgcggctggagtcctgcagaggaactgaactacacggtgccgggcccaggacctgcgggcgaagcctccccaagacagtgtaggcgctacgaggtggactggaaccagagcacctttgactgcgtggaccccctggccagcctggacaccaacaggagccgcctgccactgggcccctgccgggacggctgggtgtacgagacgcctggctcgtccatcgtcaccgagtttaacctggtatgtgccaactcctggatgttggacctattccagtcatcagtgaatgtaggattctttattggctctatgagtatcggctacatagcagacaggtttggccgtaagctctgcctcctaactacagtcctcataaatgctgcagctggagttctcatggccatttccccaacctatacgtggatgttaatttttcgcttaatccaaggactggtcagcaaagcaggctggttaataggctacatcctgattacagaatttgttgggcggagatatcggagaacagtggggattttttaccaagttgcctatacagttgggctcctggtgctagctggggtggcttacgcacttcctcactggaggtggttgcagttcacagttgctctgcccaacttcttcttcttgctctattactggtgcatacctgagtctcccaggtggctgatctcccagaataagaatgctgaagccatgagaatcattaagcacatcgcaaagaaaaatggaaaatctctacccgcctcccttcagcgcctgagacttgaagaggaaactggcaagaaattgaacccttcatttcttgacttggtcagaactcctcagataaggaaacatactatgatattgatgtacaactggttcacgagctctgtgctctaccagggcctcatcatgcacatgggccttgcaggtgacaatatctacctggatttcttctactctgccctggttgaattcccagctgccttcatgatcatcctcaccatcgaccgcatcggacgccgttacccttgggctgcatcaaatatggttgcaggggcagcctgtctggcctcagtttttatacctggtgatctacaatggctaaaaattattatctcatgcttgggaagaatggggatcacaatggcctatgagatagtctgcctggtcaatgctgagctgtaccccacattcattaggaatcttggcgtccacatctgttcctcaatgtgtgacattggtggcatcatcacgccattcctggtctaccggctcactaacatctggcttgagctcccgctgatggttttcggcgtacttggcttggttgctggaggtctggtgctgttgcttccagaaactaaagggaaagctttgcctgagaccatcgaggaagccgaaaatatgcaaagaccaagaaaaaataaagaaaagatgatttacctccaagttcagaaactagacattccattgaactaagaagagagaccgttgctgctgtcatgacctagctttgatggcagcaagaccaaaagtagaaatccctgcactcatcacaaagcccatacaactcaaccaaacttacccctgagccctatcaacctaggtctacagccagtggagtctattgtacactgtggaaaaatacccatgggaccagatcctgccaaattcttccagctcactttattctcagcattcctaggacattggacattggttttctggagggttttttttccgatctttgtatttttttaaatttgattcttttctttgcaatgctagcaaccagaatacataggggaactgtgggctaggcaaacaaaatagaaaaagtgtgaaaaacagtaaagttgggagaggagcatctattttcttaaagaaataaaacacccaaaacaaaaaaaaaaaaaatcgataccgtcgacctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 7336 bp, rat
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttggtaccgggccccccctcgaggtgggtgaagcctcaggaacagctgcctccagggaccatgtcgaccgtggatgatattctagaacatataggggaattccacctttttcagaaacaaacatttttcctcttagctctgctgtctggtgccttcacccccatctacgtgggcatcgttttcctaggatttacccctgaccaccactgctggagtcctggggcagccaagctgagccagcggtgtggctggagccaggcagaggagctgaactacaccgtgccgggtctgggaccttcggacgaggcctccttcctcagtcagtgcatgaggtatgaagtggactggaaccagagcacccttgactgtgtggacccactgtccagcctggctgcggacaggaaccagttgccattgggcccctgcgagcatggctgggtatacaacactcccggctcctccattgtcactgagtttaacctggtgtgtgctcactcctggatgctggacctgtttcagtcagtagtgaacgtggggtttttcatcggtgctatgatgattggctacctagcggacaggtttggccggaagttctgcctcttggtcaccatcctcatcaatgctatctctggggctctcatggcgatttctcccaactatgcctggatgttggtgtttcgctttctccagggactggtcagcaaagcaggctggttaattggctacatcctgattacagaatttgttgggctgggctatcgcagaatggtggggatttgctatcaaatcgccttcaccgttggcctcctgatcctggctggagtggcctatgtgattcccaactggagatggctacaatttgctgtgactctgcccaacttctgcttcctgctctatttctggtgcataccggaatctccaagatggctgatctcccagaacaaaatcgtaaaagccatgaagataattaaacatattgctaagaaaaatggaaagtcggtgccggtctctcttcagaacctgacaccagatgaggacgctggcaagaaattgaacccttcattcctggacttggtcagaacccctcagataaggaaacacacgttgatcttgatgtacaattggttcacgagctctgttctctaccagggcctcatcatgcacatgggccttgcaggggacaacatctacctggatttcttctactccgccctggtggagtttccagccgccttcatcatcatcctcaccatcgaccgggttggtcgccgctatccctgggctgtgtcaaatatggtggcaggagcagcctgtctagcctctgtttttatccctgatgatctacagtggctgaaaattaccattgcatgcttgggtagaatgggcatcaccatggcctatgaaatggtctgcctggtcaatgctgagctgtaccccacatacatcaggaatcttggtgtccttgtctgctcctccatgtgtgacattggcggcatcatcacgcctttcctcgtctaccgtctcacggacatctggatggagttcccactggttgtatttgctgtggttggccttgtcgctggggcacttgtgctgttgctacctgagaccaaagggaaggctctgcctgagaccatcgaggatgccgagaatatgcagaggccaagaaaaaaagaaagaaaagagaatttacctccaagtcaagcaagcagaccgtccgctaagctaaaaagaaagggcatcattgctgctggagctgactttgctctctctgaggccagagatggagcttctctctcccctccccccaaacccacacaaaccaacctcacttacccctgaactccatcagcaagagctgtagcttgcacggtctgttgcactgatgtgtcaagctcttcctcccagccaggattttccgcctcactctgctctcagcactcccaggaaatgaccattgctttactggattcgttatggttgttttttcatctttacattctttatttagtttccttctccaccacaacatcaaaccaaaatacccaagggagctgtgggccaggacaaccaactattaaagaagtaagaaatctgaaaaaacaataaacgtgggagaagagaaaaaaaaaaaaaaaaaaaaaaaaaaaaaaaaaaaaaaaactcgaggtcgacggtatcgataagcttgatatcgaattcctgcagcccgggggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 3 - DNA, 6894 bp, Homo sapiens
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttgctgagccatcatgcccaccgtggatgacattctggagcaggttggggagtctggctggttccagaagcaagccttcctcatcttatgcctgctgtcggctgcctttgcgcccatctgtgtgggcatcgtcttcctgggtttcacacctgaccaccactgccagagccctggggtggctgagctgagccagcgctgtggctggagccctgcggaggagctgaactatacagtgccaggcctggggcccgcgggcgaggccttccttggccagtgcaggcgctatgaagtggactggaaccagagcgccctcagctgtgtagaccccctggctagcctggccaccaacaggagccacctgccgctgggtccctgccaggatggctgggtgtatgacacgcccggctcttccatcgtcactgagttcaacctggtgtgtgctgactcctggaagctggacctctttcagtcctgtttgaatgcgggcttcttctttggctctctcggtgttggctactttgcagacaggtttggccgtaagctgtgtctcctgggaactgtgctggtcaacgcggtgtcgggcgtgctcatggccttctcgcccaactacatgtccatgctgctcttccgcctgctgcagggcctggtcagcaagggcaactggatggctggctacaccctaatcacagaatttgttggctcgggctccagaagaacggtggcgatcatgtaccagatggccttcacggtggggctggtggcgcttaccgggctggcctacgccctgcctcactggcgctggctgcagctggcagtctccctgcccaccttcctcttcctgctctactactggtgtgtgccggagtcccctcggtggctgttatcacaaaaaagaaacactgaagcaataaagataatggaccacatcgctcaaaagaatgggaagttgcctcctgctgatttaaagatgctttccctcgaagaggatgtcaccgaaaagctgagcccttcatttgcagacctgttccgcacgccgcgcctgaggaagcgcaccttcatcctgatgtacctgtggttcacggactctgtgctctatcaggggctcatcctgcacatgggcgccaccagcgggaacctctacctggatttcctttactccgctctggtcgaaatcccgggggccttcatagccctcatcaccattgaccgcgtgggccgcatctaccccatggccgtgtcaaatttgttggcgggggcagcctgcctcgtcatgatttttatctcacctgacctgcactggttaaacatcataatcatgtgtgttggccgaatgggaatcaccattgcaatacaaatgatctgcctggtgaatgctgagctgtaccccacattcgtcaggaacctcagagtgatggtgtgttcctccctgtgtgacataggtgggataatcacccccttcatagtcttcaggctgagggaggtctggcaagccttgcccctcattttgtttgcggtgttgggcctgcttgccgcgggagtgacgctacttcttccagagaccaagggggtcgctttgccagagaccatgaaggacgccgagaaccttgggagaaaagcaaagcccaaagaaaacacgatttaccttaaggtccaaacctcagaaccctcgggcacctgagagagatgttttgcggcgatgtcgtgttggagggatgaagatggagttatcctctgcagaaattcctagacgccttcacttctctgtattcttcctcatacttgcctacccaagccgaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 4176 bp, Homo sapiens
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacacattgatgagcaatgcttttttataatgccaactttgtacaaaaaagcaggcttcatgaccttctcggagatcctggaccgtgtgggaagcatgggccatttccagttcctgcatgtagccatactgggcctcccgatcctcaacatggccaaccacaacctgctgcagatcttcacagccgccacccctgtccaccactgtcgcccgccccacaatgcctccacagggccttgggtgctccccatgggcccaaatgggaagcctgagaggtgcctccgttttgtacatccgcccaatgccagcctgcccaatgacacccagagggccatggagccatgcctggatggctgggtctacaacagcaccaaggactccattgtgacagagtgggacttggtgtgcaactccaacaaactgaaggagatggcccagtctatcttcatggcaggtatactgattggagggctcgtgcttggagacctgtctgacaggtttggccgcaggcccatcctgacctgcagctacctgctgctggcagccagcggctccggtgcagccttcagccccaccttccccatctacatggtcttccgcttcctgtgtggctttggcatctcaggcattaccctgagcaccgtcatcttgaatgtggaatgggtgcctacccggatgcgggccatcatgtcgacagcactcgggtactgctacacctttggccagttcattctgcccggcctggcctacgccatcccccagtggcgttggctgcagttaactgtgtccattcccttcttcgtcttcttcctatcatcctggtggacaccagagtccatacgctggttggtcttgtctggaaagtcctcgaaggccctgaagatactccggcgggtggctgtcttcaatggcaagaaggaagagggagaaaggctcagcttggaggagctcaaactcaacctgcagaaggagatctccttggccaaggccaagtacaccgcaagtgacctgttccggatacccatgctgcgccgcatgaccttctgtctttccctggcctggtttgctaccggttttgcctactatagtttggctatgggtgtggaagaatttggagtcaacctctacatcctccagatcatctttggtggggtcgatgtcccagccaagttcatcaccatcctctccttaagctacctgggccggcataccactcaggccgctgccctgctcctggcaggaggggccatcttggctctcacctttgtgcccttggacttgcagaccgtgaggacagtattggctgtgtttgggaagggatgcctatccagctccttcagctgcctcttcctctacacaagtgaattataccccacagtcatcaggcaaacaggtatgggcgtaagtaacctgtggacccgcgtgggaagcatggtgtccccgctggtgaaaatcacgggtgaggtacagcccttcatccccaatatcatctacgggatcaccgccctcctcgggggcagtgctgccctcttcctgcctgagaccctgaatcagcccttgccagagactatcgaagacctggaaaactggtccctgcgggcaaagaagccaaagcaggagccagaggtggaaaaggcctcccagaggatccctctacagcctcacggaccaggcctgggctccagctaggacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaataatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 5 - DNA, 4206 bp, Homo sapiens
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttacaacgccaactttgtacaaaaaagcaggcttaggaatgcgggactacgacgaggtgatcgccttcctgggcgagtgggggcccttccagcgcctcatcttcttcctgctcagcgccagcatcatccccaatggcttcaatggtatgtcagtcgtgttcctggcggggaccccggagcaccgctgtcgagtgccggacgccgcgaacctgagcagcgcctggcgcaacaacagtgtcccgctgcggctgcgggacggccgcgaggtgccccacagctgcagccgctaccggctcgccaccatcgccaacttctcggcgctcgggctggagccggggcgcgacgtggacctggggcagctggagcaggagagctgcctggatggctgggagttcagccaggacgtctacctgtccaccgtcgtgaccgagtggaatctggtgtgtgaggacaactggaaggtgcccctcaccacctccctgttcttcgtaggcgtgctcctcggctccttcgtgtccgggcagctgtcagacaggtttggcaggaagaacgttctcttcgcaaccatggctgtacagactggcttcagcttcctgcagattttctccatcagctgggagatgttcactgtgttatttgtcatcgtgggcatgggccagatctccaactatgtggtagccttcatactaggaacagaaattcttggcaagtcagttcgtattatattctctacattaggagtgtgcacattttttgcagttggctatatgctgctgccactgtttgcttacttcatcagagactggcggatgctgctgctggcgctgacggtgccgggagtgctgtgtgtcccgctgtggtggttcattcctgaatctccccgatggctgatatcccagagaagatttagagaggctgaagatatcatccaaaaagctgcaaaaatgaacaacatagctgtaccagcagtgatatttgattctgtggaggagctaaatcccctgaagcagcagaaagctttcattctggacctgttcaggactcggaatattgccataatgaccattatgtctttgctgctatggatgctgacctcagtgggttactttgctctgtctctggatgctcctaatttacatggagatgcctacctgaactgtttcctctctgccttgattgaaattccagcttacattacagcctggctgctattgcgaacgctgcccaggcgttatatcatagctgcagtactgttctggggaggaggtgtgcttctcttcattcaactggtacctgtggattattacttcttatccattggtctggtcatgctgggaaaatttgggatcacctctgctttctccatgctgtatgtcttcactgctgagctctacccaaccctggtcaggaacatggcggtgggggtcacatccacggcctccagagtgggcagcatcattgccccctactttgtttacctcggtgcttacaacagaatgctgccctacatcgtcatgggtagtctgactgtcctgattggaatcctcaccctttttttccctgaaagtttgggaatgactcttccagaaaccttagagcagatgcagaaagtgaaatggttcagatctgggaaaaaaacaagagactcaatggagacagaagaaaatcccaaggttctaataactgcattctgagacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 6 - DNA, 6870 bp, Homo sapiens
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttggtaccgagctcggatccactagtccagtgtggtggaattctgcagatatcacaagtttgtacaaaaaagcaggcaccatgcgggactacgacgaggtgaccgccttcctgggcgagtgggggcccttccagcgcctcatcttcttcctgctcagcgccagcatcatccccaatggcttcaccggcctgtcctccgtgttcctgatagcgaccccggagcaccgctgccgggtgccggacgccgcgaacctgagcagcgcctggcgcaaccacactgtcccactgcggctgcgggacggccgcgaggtgccccacagctgccgccgctaccggctcgccaccatcgccaacttctcggcgcttgggctggagccggggcgcgacgtggacctggggcagctggagcaggagagctgtctggatggctgggagttcagtcaggacgtctacctgtccaccattgtgaccgagtggaacctggtgtgtgaggacgactggaaggccccactcacaatctccttgttcttcgtgggtgtgctgttgggctccttcatttcagggcagctgtcagacaggtttggccggaagaatgtgctgttcgtgaccatgggcatgcagacaggcttcagcttcctgcagatcttctcgaagaattttgagatgtttgtcgtgctgtttgtccttgtaggcatgggccagatctccaactatgtggcagcatttgtcctggggacagaaattcttggcaagtcagttcgtataatattctctacgttaggagtgtgcatattttatgcatttggctacatggtgctgccactgtttgcttacttcatccgagactggcggatgctgctggtggcgctgacgatgccgggggtgctatgcgtggcactctggtggttcatccctgagtccccccgatggctcatctctcagggacgatttgaagaggcagaggtgatcatccgcaaggctgccaaagccaatgggattgttgtgccttccactatctttgacccgagtgagttacaagacctaagttccaagaagcagcagtcccacaacattctggatctgcttcgaacctggaatatccggatggtcaccatcatgtccataatgctgtggatgaccatatcagtgggctattttgggctttcgcttgatactcctaacttgcatggggacatctttgtgaactgcttcctttcagcgatggttgaagtcccagcatatgtgttggcctggctgctgctgcaatatttgccccggcgctattccatggccactgccctcttcctgggtggcagtgtccttctcttcatgcagctggtacccccagacttgtattatttggctacagtcctggtgatggtgggcaagtttggagtcacggctgccttttccatggtctacgtgtacacagccgagctgtatcccacagtggtgagaaacatgggtgtgggagtcagctccacagcatcccgcctgggcagcatcctgtctccctacttcgtttaccttggtgcctacgaccgcttcctgccctacattctcatgggaagtctgaccatcctgacagccatcctcaccttgtttctcccagagagcttcggtaccccactcccagacaccattgaccagatgctaagagtcaaaggaatgaaacacagaaaaactccaagtcacacaaggatgttaaaagatggtcaagaaaggcccacaatccttaaaagcacagccttctagaacccagctttcttgtacaaagtggtgatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

(19) United States
(12) Patent Application Publication
(10) Pub. No.: US 2009/0184006 A1
(43) Pub. Date: Jul. 23, 2009

(54) METHOD FOR THE DETERMINATION OF THE ACTIVITY OF THE ORGANIC CATION TRANSPORTER

(75) Inventors: Henning Vollert, Bad Segeberg (DE); Sven Geibel, Frankfurt am Main (DE); Bela Kelety, Frankfurt am Main (DE); Klaus Fendler, Frankfurt (DE); Petra Arndt, Frankfurt am Main (DE); Olga Gaiko, Frankfurt (DE); Ingo Janausch, Frankfurt (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/162,833

(22) PCT Filed: Jan. 22, 2007
(86) PCT No.: PCT/EP2007/000497

§ 371 Date: Dec. 12, 2008

(30) Foreign Application Priority Data
Jan. 31, 2006 (EP) 06001906.4

(51) Int. Cl. G01N 33/50; G01N 27/26

(52) U.S. Cl. 205/792; 204/403.01

(57) ABSTRACT

The present invention refers to a method for determining the activity of the organic cation transporter (OCT), a method for determining the activity of or identifying a chemical compound that modulates the activity of OCT with the help of a cell free electrophysiological sensor chip containing a solid-supported sensor electrode and a lipid layer containing the OCT located in the immediate spatial vicinity to the sensor electrode, whereas the sensor electrode is electrically insulated relative to the solutions used and to the lipid layer, as well as to the sensor chip itself and a kit containing same.

The present invention refers to a method for determining the activity of the organic cation transporter (OCT), a method for determining the activity of or identifying a chemical compound that modulates the activity of OCT with the help of a cell free electrophysiological sensor chip containing a solid-supported sensor electrode and a lipid layer containing the OCT located in the immediate spatial vicinity to the sensor electrode, whereas the sensor electrode is electrically insulated relative to the solutions used and to the lipid layer, as well as to the sensor chip itself and a kit containing same.

The human organic cation transport is an important mechanism for the transcellular transport of organic cations. Therefore, the organic cation transporters (OCTs) are not only potential drug targets that allow direct influence on disease-related abnormalities, but also potential ADMET (Adsorption, Distribution, Metabolism, Excretion and Toxicity) targets allowing for alterations of bioavailiblity parameters of potential drugs.

The OCT belongs to a superfamily that includes uniporters, symporters and antiporters, such as multidrug-resistance proteins, facilitative diffusion systems and proton antiporters. They mediate transport of small cations with different molecular structures independently of sodium and proton gradients. Substrate-specific, sodium independent transport mechanisms via the human OCT (hOCT) have been described in liver, kidney, small intestine and the nervous system (Pritchard J B & Miller D S (1993), Physiol. Rev. 73 (4) 765-796). The human organic cation transporter hOCT1 has already been cloned in 1997 (Zhang, L. et. al. (1997) Mol. Pharmacology 51 (6), 913-921).

The OCT shifts electrical charges while going through its transport cycle. This shift may originate either from the movement of charged substrates or from the movement of protein moieties carrying (partial) charges. Activities of OCTs can be monitored via radiofluxes and standard two electrode voltage clamp electrophysiology with the common drawbacks of either method as bad time resolution, low sensitivity, difficult discrimination between blockers and competitive substrates, false positives and negatives etc. (Arndt et al. (2001) Am J Physiol Renal Physiol, 281, F454-F468).

In some other cases the transporter-related currents can either be directly monitored in a rather physiological environment by patch-clamp experiments or at artificial “black lipid membranes”. In the latter case, a lipid bilayer is generated in a small hole between two buffer reservoirs, each of them containing an Ag/AgCl electrode. After incorporation of the protein into the bilayer, the biological activity (e.g. enzymatic activity) can be triggered e.g. by photoactivation of ATP derivatives. Yet, due to its lack of stability, no rapid buffer exchange experiments can be conducted with this system, limiting the system to photoactivatable substrates. The lack of stability can be overcome by immobilizing protein-containing particles on a sensor surface or sensor chip.

A cell free electrophysiological sensor chip is generally based on transporter-containing membrane fragments or vesicles usually electrically coupled to a gold coated biochip. The membrane fragments usually adsorb to the sensor chip surface which preferably carries a modified lipid layer on a thin gold film. The membrane fragments can generally form cavities that are able to maintain ion gradients across the membranes. After the activation with a suitable substrate, ions or charged substrates are transported across the membrane. Since both the adsorbed membrane fragments and the covered electrode surface behave like electrical capacitors, ions in motion represent a changing current that becomes detectable if a reference electrode is placed in the surrounding solution.

The problem of the present invention concerns the question whether the activity of the OCT can specifically and sensitively be detected with such a sensor chip although patch clamp experiments with hOCT1 failed.

Surprisingly it has been found that a cell-free assay could be established which showed the required sensitivity in order to detect a specific signal upon the activation of OCT. It was particularly surprising because OCT functioned in the cell-free assay according to the present invention without the cellular background, i.e. without intracellular substances, the cytoskeleft etc. In particular, the assay of the present invention can be carried out in a broad pH and/or high ion concentration range which is of particular advantage.

Consequently, a first embodiment of the present invention refers to a method for determining the activity of OCT with the following consecutive steps:
(a) providing a cell free electrophysiological sensor chip containing a solid-supported sensor electrode and a lipid layer containing the OCT located in the immediate spatial vicinity to the sensor electrode, whereas the sensor electrode is electrically insulated relative to the solutions used and to the lipid layer,
(b) treating the sensor chip with an ion-containing non-activating solution,
(c) treating the sensor chip with an ion- and substrate containing activating solution, and
(d) measuring the electric signal.

The OCT is, for example, selected from SLC22A1 (OCT1), SLC22A2 (OCT2, SLC22A3 (OCT3), SLC22A4 (OCTN1), and SLC22A5 (OCTN2). Usually it is of mammalian origin, particularly from rat, mouse, rabbit, pig, guinea pig, drosophila melanogaster, caenorhabditis elegans or human. Preferably it is human OCT1.

The electrode usually comprises a metallic material or an electrically conductive metal oxide, particularly gold, platinum, silver or indium tin oxide.

The solid-supported sensor electrode is generally a glass- or a polymer-supported sensor electrode, in particular a borofloat-glass-supported sensor electrode, particularly a borofloat-glass-supported gold electrode. In a preferred embodiment the lipid layer is attached to the electrode via a chemical bond, particularly via his-tag coupling or streptavidin-biotin coupling, or via hydrophobic, hydrophilic or ionic forces.

The electrode is further electrically insulated, for example, by one or more insulating monolayer(s), particularly by one or more insulating amphiphilic organic compounds, more particularly by one or more insulating membrane monolayer(s), most particularly by a mercaptan layer, especially octadecyl thiol, as an under layer facing the electrode and a membrane monolayer as an upper layer facing away from the electrode.

A sensor chip especially contains a solid support carrying the sensor electrode and a cover plate with a hole, forming a well similar to those of titer plates. Either glass or polymer plates serve as suitable supports. In the case of a glass support, e.g. a glass plate, the electrode preferably contains a thin, lithographically structured gold film, which has been chemically modified, e.g. by means of a mercaptane, on its surface, whereas with a polymer support modified thick film gold electrodes can also be used. Due to the range of suitable substrates, single sensor chips can be manufactured as well as sensor strips or even sensor array plates with 96 or 384 sensors. Particularly the polymer-based sensors bear the potential for low cost mass production.

Generally for all sensor types the gold surface is turned into a capacitor after the surface modification has taken place and the well has been filled with an aqueous solution. The properties of this capacitor can be determined by the aid of a current-carrying reference electrode such as Pt/Pt or Ag/AgCl, indium tin oxide or others brought in contact with the solution. Furthermore, the sensor surface is preferably very hydrophilic, i.e. sticky for membrane fragments and vesicles. Consequently, the OCT kept within its native or native-like environment, e.g. in biological membrane sheets, vesicles or proteoliposomes readily adsorbs to the hydrophilic sensor surface, forming compartments whose inner space with its solution is electrically isolated from both, the gold surface as well as the surrounding solution within the well. If inserted into a cuvette, the well of the chip defines the inner volume of a flow cell, enabling a rapid solution exchange above the sensor surface.

A cell free electrophysiological sensor chip used for the present invention is for example described in WO02/074983, in particular in the claims and/or FIGS. 1 and/or 2 including the description of the figures of said PCT application, which is hereby incorporated by reference, if not otherwise described in the present invention. It is also available from IonGate Biosciences GmbH, Frankfurt/Main, Germany sold under the name SURFE²R ONE® biosensor system.

If one switches from a solution which does not contain a substrate or activator of the OCT to a solution that does, a measurable, transient charging current of the electrode is induced which is typically within the range of 100 pA to 4 nA. Therefore, replacement of the non-activating solution by the activating solution, i.e. the substrate-containing solution, will trigger the OCT activity. Replacing the solutions subsequently in reverse order returns the sensor chip into its initial state. According to the present invention a particular advantage of the ion-containing solutions is that artifacts are minimized which leads to a specific and sensitive signal.

All components necessary for carrying out solution exchange experiments can be accommodated in a PC- or otherwise controlled workstation. In the conventional system, the non-activating (i.e. substrate-free) solution and the activating solution are generally stored in glass bottles. Air pressure usually applied to the bottles drives the solution through a system of electromechanically operated valves and through the flow cell. Alternatively, an auto sampler can be used to process several solutions in an automated fashion.

Prior to the use of the sensor chip it is preferred to wash the electrode with an ion-containing washing solution.

In any case the ion-containing solutions of the present invention preferably contain univalent and bivalent ions selected from Na⁺, K⁺, Mg²⁺ and/or Ca²⁺.

The total concentration of the ions in the ion-containing solutions is preferably from about 100 mM to about 1000 mM, particularly from about 200 mM to about 500 mM, more particularly from about 300 mM to about 500 mM, most particularly about 435 mM. The concentration of the univalent ions in the ion-containing solutions is preferably from about 300 mM to about 400 mM and the concentration of the bivalent ions in the ion-containing solutions is preferably from about 2 mM to about 10 mM, particularly from about 5 mM to about 8 mM, more particularly about 5 mM.

In another preferred embodiment the ion-containing solutions further contain a buffer, particularly a HEPES/NMG, 30±10 mM, pH 7.0±1.0 buffer.

Examples of the ion-containing solutions are for

### (a) A Washing Solution:

30±10 mM of a buffer, e.g. HEPES/NMG, pH 7.0±1.0,
300±100 mM of a univalent ion, e.g. NaCl,
4±2 mM of a bivalent ion, e.g. MgCl₂.

### (b) A Non-Activating Solution:

30±10 mM of a buffer, e.g. HEPES/NMG, pH 7.0±1.0,
300±100 mM of a univalent ion, e.g. NaCl,
4±2 mM of a bivalent ion, e.g. MgCl₂, and
0.5-100 mM of a univalent ion, e.g. NaCl, which should be equimolar to the concentration of the substrate in the activating solution.

### (c) An Activating Solution:

30±10 mM of a buffer, e.g. HEPES/NMG, pH 7.0±1.0,
300±100 mM of a univalent ion, e.g. NaCl,
4±2 mM of a bivalent ion, e.g. MgCl₂, and
0.5-100 mM of a substrate, e.g. choline chloride.

The substrate of the activating solution is generally an organic cation, particularly a cationic drug, a cationic xenobiotic and/or a cationic vitamin, more particularly a primary, secondary, tertiary or quaternary amine, most particularly choline, acetylcholine, nicotine, N¹-methylnicotineamide, morphine, 1-methyl-4-phenylpyridinium, procainamide, tetraethylammonium, tributylmethylammonium, debrisoquine or a biogenic amine like epinephrine, norpeinephrine or carnitine or lipophilic compounds like quinine, quinidine or steroids like corticosterone or organic anions like para-amino hippuric acid, probenecid.

In general, the electric signal is measured using amperometric and/or potentiometric means, and the steps (b) to (d) are carried out at least 2 times, particularly 2 to 4 times.

The term “electric signal” or “current” in context of this invention shall mean the peak current in response to the replacement of non-activating by activating solution, including but not limited to the maximal peak current. The current amplitude rises usually within 10 to 100 ms, followed by a slower decay within about 2 seconds. The polarity of the current may be positive or negative, depending on the polarity of the transported ions and/or the polarity of the shifted moieties of the protein and the vectorial orientation of their transport or shift across or within the membranes of the compartments. Currents resulting from the replacement of the activating solution by non-activating solution or from the replacement of the non-activating solution by the washing solution are generally not taken into consideration with respect to the determination of the OCT activity. Flow rates and intervals are preferably chosen such that the current response to the replacement of the non-activating solution by activating solution remains unbiased by current responses provoked by the other replacement steps.

The method of the present invention can also be carried out in the presence of a chemical compound, particularly a stimulator (activator) or an inhibitor of OCT.

Therefore, the present invention also refers to a method for identifying a chemical compound that modulates the activity of OCT with the following consecutive steps:
(a) carrying out the method of the present invention, and
(b) identifying the chemical compound.

The chemical compound is generally an organic cation, particularly a cationic drug, a cationic xenobiotic and/or a cationic vitamin and/or biogenic amines, more particularly a primary, secondary, tertiary or quaternary amine, wherein the chemical compound usually is a stimulator or an inhibitor of OCT. The chemical compound can for example be present in a chemical compound library.

Another subject-matter of the present invention is the cell free electrophysiological sensor chip itself containing the OCT, as described above in detail. The OCT is bound to the sensor chip according to methods generally known to a person skilled in the art and/or as specifically described in the Example.

The sensor chip can further comprise a data acquisition device for acquiring measurement data from the electrode, and optionally exchange and/or mixing means for making available exchanging and/or mixing the ion-containing solutions. The sensor chip can also be in the form of a microplate or microtiter plate.

Another subject-matter of the present invention is an apparatus containing a sensor chip of the present invention, a reference electrode, a data acquisition device for acquiring measurement data from the electrode, an exchange and/or mixing means for making available exchanging and/or mixing the ion-containing solutions, a flow analysis device, a power supply, a computer and an autosampler. The reference electrode is preferably a Pt/Pt, Ag/AgCl or indium tin oxide electrode.

A further subject-matter of the present invention is a kit containing
(a) a cell free electrophysiological sensor chip of the present invention or an apparatus of the present invention,
(b) at least one ion-containing solution as defined above, and optionally
(c) a substrate as defined above.

The following Figures, Tables, Sequences and Examples shall explain the present invention without limiting the scope of the invention.

### DESCRIPTION OF THE FIGURES

FIG. 1A shows electrical responses of a typical sensor with immobilized membranes harbouring rOCT2 (slc22a2) upon addition of activating solution (30 mM CholineCl) before (black trace) and after inhibition (grey trace) with 1 mM TBA.

FIG. 1B shows electrical responses of a typical sensor with immobilized membranes harbouring hOCT2 (SLC22A1) upon addition of activating solution (30 mM CholineCl) before (black trace) and after inhibition (grey trace) with 1 mM TBA.

FIG. 2A shows choline concentration dependence of rOCT2 (slc22a2) (CHO cell membranes).

FIG. 2B shows choline concentration dependence of hOCT2 (SLC22A1) (CHO cell membranes).

FIG. 3 shows the pH dependence of rOCT2 (slc22a2) and hOCT2 (SLC22A2) from insect cells.

FIG. 4A shows the IC50 of TBA of rOCT2 (slc22a2) (CHO cells). IC50 was determined using 10 mM choline as a substrate.

FIG. 4B shows the IC50 of TBA of hOCT2 (SLC22A2) (CHO cells). IC50 was determined using 30 mM choline as a substrate.

FIG. 5A shows electrical current of stably expressed rOCT2 (slc22a2) in patch clamp experiments (CHO cells).

FIG. 5B shows electrical current of stably expressed hOCT2 (slc22a2) in patch clamp experiments (CHO cells).

FIG. 6A shows the IC50 of quinine of rOCT2 (slc22a2) (CHO cells). IC50 was determined using 10 mM choline as a substrate.

FIG. 6B shows the acetylcholine concentration dependence of rOCT2 (slc22a2) (CHO cells).

FIG. 7 shows a nucleic acid sequence containing the coding region of human OCT2 (hOCT2, SLC22A2)). The start (ATG) and stop (TAA) sites of the gene are in bold face and underlined. The XhoI/XhoI (CTCGAG) cloning sites are underlined.

FIG. 8 shows a nucleic acid sequence containing the coding region of rat OCT2 (rOCT2; slc22a2). The start (ATG) and stop (TGA) sites of the gene are in bold face and underlined. The KpnI (GGTACC) and BamHI (GGATCC) cloning sites are underlined.

FIG. 9 shows a nucleic acid sequence containing the coding region of human OCT1 (hOCT1; SLC22A1). The start (ATG) and stop (TGA) sites of the gene are in bold face and underlined. The HINDIII (AAGCTT) and EcoRV (GATATC) cloning sites are underlined.

FIG. 10 shows a nucleic acid sequence containing the coding region of human OCT3 (hOCT3; SLC22A3). The start (ATG) and stop (TAG) sites of the gene are in bold face and underlined.

FIG. 11 shows a nucleic acid sequence containing the coding region of human OCTN1 (SLC22A4). The start (ATG) and stop (TGA) sites of the gene are in bold face and underlined.

FIG. 12 shows a nucleic acid sequence containing the coding region of human OCTN2 (SLC22A5). The start (ATG) and stop (TAG) sites of the gene are in bold face and underlined.

### DESCRIPTION OF THE SEQUENCES

SEQ ID NO: 1 shows a nucleic acid sequence containing the coding region of human OCT2 (hOCT2; SLC22A2).
SEQ ID NO: 2 shows a nucleic acid sequence containing the coding region of rat OCT2 (rOCT2; slc22a2)).
SEQ ID NO: 3 shows a nucleic acid sequence containing the coding region of human OCT1 (hOCT1; SLC22A3).
SEQ ID NO: 4 shows a nucleic acid sequence containing the coding region of human OCT3 (hOCT3; SLC22A3).
SEQ ID NO: 5 shows a nucleic acid sequence containing the coding region of human OCTN1 (SLC22A4).
SEQ ID NO: 6 shows a nucleic acid sequence containing the coding region of human OCTN2 (SLC22A5).

### EXAMPLES

### Materials

| Washing solution (C): | HEPES/NMG | 30 mM, pH 7.4 | |
|---|---|---|---|
| | NaCl | 300 | mM |
| | MgCl₂ | 5 | mM |
| Non-activating solution (B): | HEPES/NMG | 30 mM, pH 7.4 | |
| | NaCl | 400 | mM |
| | MgCl₂ | 5 | mM |
| Activating solution (A): | HEPES/NMG | 30 mM, pH 7.4 | |
| | NaCl | 300 | mM |
| | Choline/Cl | 100 | mM |
| | MgCl₂ | 5 | mM |
| In solution C, B and A, | TBA or Quinine | 10 | μM; |
| respectively | | | |

### Assay Procedure

### (a) Preparation of Membranes

After harvesting the cells from a virally transfected Sf9 or HighFive suspension cell line or an stably transfected adherent CHO cell line via centrifugation, aliquots of approx. 2 g wet weight cells were quick-frozen in liquid nitrogen and stored at −80° C. for further preparation.

The cell pellet was thawed on ice and transferred to ice-cold buffer (0.25 M sucrose, 5 mM Tris pH 7.5, 2 mM DTT, one complete protease inhibitor cocktail tablet per 50 ml (Roche Diagnostics GmbH, Mannheim, Germany).

The membrane fragments were prepared by cell rapture. Cells were homogenized by the nitrogen cell disruption method utilizing a Parr Cell Disruption Bomb (Parr Instrument Company, Illinois, USA) or the Dounce homogenisation method utilizing a Dounce Homogenisator (7 ml from Novodirect GmbH, Kehl/Rhein, Germany) and the suspension centrifuged 10 min at 4° C. and 680 g and 10 min at 4° C. and 6100 g. The supernatants were collected and again centrifuged for 1 h at 4° C. and 100,000 g in SW41 swing-out rotor.

Pellets were suspended in approximately 2 ml of 5 mM Tris pH 7.5. With 87% sucrose (in 5 mM Tris) the suspension was adjusted to 56%. The sucrose gradient was then built up beginning with 2 ml of the 56% fraction at the bottom, following 3 ml 45% sucrose, 3 ml 35% and 2 ml 9% sucrose.

Again centrifugation for 2.5 h (or even more) at 4° C. and 100000 g the gradient-bands were aspirated carefully with a pasteur pipette and collected in fresh tubes together with either 5 ml of 300 mM NaCl, 5 mM MgCl₂, 30 mM Hepes pH 7.5 or 10 mM Tris/HCl pH7.5.

Another centrifugation step followed: 1 h at 150000 g, 4° C.

The resulting pellet was resuspended in 300 mM NaCl, 5 mM MgCl₂, 2 mM DTT, 30 mM Hepes pH 7.5, 10% glycerol.

### (b) Preparation of Biosensors

Biosensors were prepared according to the following protocol.
1. Addition of 30 μl mercaptane solution (2% mercaptane in isopropanol) to biosensor
2. Incubation time: 15 min
3. Rinsing with 3×70 μl isopropanol
4. Vacuum dry biosensor
5. Drying time: 30 min
6. Addition of 2 μl lipid (60 units (weight) 2-Diphytanoyl-sn-Glycero-3-Phosphocholin+1 unit octadecylamine dissolved in 800 units n-decane)
7. Immediate addition 30 μl DTT-Buffer (1,542 mg DTT/50 ml Buffer C)
8. Incubation time: 20 min.
9. Addition of 20 μl membrane preparation+135 μl DTT-Buffer C and Mixing (for 6 sensors)
10. Sonication: 2×10 times (settings 0.5 s/30%) with a pause on ice of 30 s
11. Removal of buffer from biosensor
12. Immediate addition of 25 μl membrane solution to the biosensors (mix 3 times)
13. Store overnight in the refrigerator (in Petri dish with high humidity)

### (c) Solution Exchange Protocol

For the determination of its activity, the OCT protein was treated consecutively with a washing, non-activating and activating solution and the electrical current was measured when changing from charging to activating treatment. The replacement of the washing and non-activating solution by activating solution (substrate containing solution) triggers the OCT activity. Subsequently replacing solutions in reverse order returns the sensor chip into its initial state.

### Cycle 1:

| non- | | non- |
|---|---|---|
| activating | activating | activating |
| solution | solution | solution |
| 4 s | 4 s | 4 s |
| 4 s | 4 s | 4 s |
| 4 s | 4 s | 4 s |

1 minutes break

### Cycle 2:

| non- | | non- |
|---|---|---|
| activating | activating | activating |
| solution | solution | solution |
| 4 s | 4 s | 4 s |
| 4 s | 4 s | 4 s |
| 4 s | 4 s | 4 s |
| 4 s | 4 s | 4 s |
| 4 s | 4 s | 4 s |
| 4 s | 4 s | 4 s |

5 minutes break and addition of a compound to be analyzed

### Cycle 3:

| non- | | non- |
|---|---|---|
| activating | activating | activating |
| solution | solution | solution |
| 4 s | 4 s | 4 s |
| 4 s | 4 s | 4 s |
| 4 s | 4 s | 4 s |

1 minutes break

### Cycle 4:

| non- | | non- |
|---|---|---|
| activating | activating | activating |
| solution | solution | solution |
| 4 s | 4 s | 4 s |
| 4 s | 4 s | 4 s |
| 4 s | 4 s | 4 s |
| 4 s | 4 s | 4 s |
| 4 s | 4 s | 4 s |
| 4 s | 4 s | 4 s |

5 minutes break and addition of the same compound in another concentration or of another compound, etc.

The following settings were used for the measurements of hOCT2:

After buffer containers A, B, and C of the biosensor system had been filled with “activating” buffer and “non-activating” buffer a dummy was mounted to the sensor holder and the system was flushed with all buffers to remove air bubbles from the entire fluidic system. An empty or blind sensor was then replaced by a standard glass-based sensor preloaded with hOCT2-containing CHO membrane fragments (chemically modified gold surface of 3 mm diameter; IonGate Biosciences GmbH, Frankfurt/M., Germany). Liquid transport through the fluidic system, including the sensor flow cell, was achieved by applying air pressure to the buffer containers.

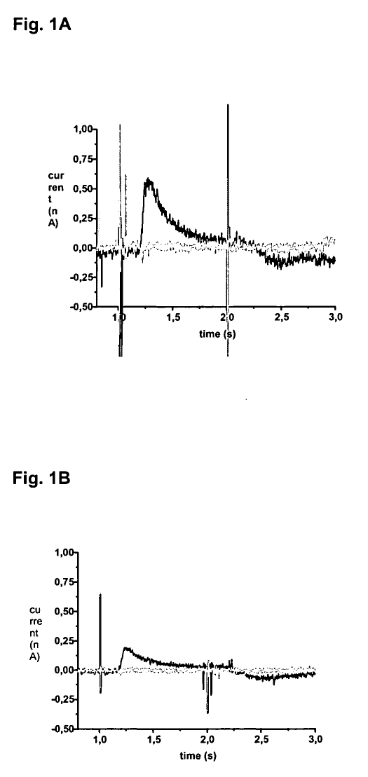

Measurements were usually carried out at 250 mbar overpressure, resulting in a flow rate of about 300 μl s⁻¹. For the determination of its activity, the membranes harboring OCT protein were treated consecutively by a “non-activating” and “activating” solution. Subsequently replacing solutions in reverse order returns the sensor chip into its initial state. By means of the control software, a sequence was defined (see FIG. 1), in which “non-activating” buffer flowed over the sensor surface, followed by “activating” buffer and “non-activating” buffer. During the whole sequence, the current response was digitized (2000 samples s⁻¹) and saved to data files. For dose-response experiments inhibitors were dissolved in “non-activating” and “activating” buffer, respectively. All chemicals were of analytical grade or better.

### Data Analysis

High control: electrical valley current after activation with 100 mM choline/Cl before inhibition;
Low control: electrical valley current after activation with 0 mM choline/Cl after inhibition;

Results are calculated from the corrected raw data.

$$Inhibition of transporter = 100 * ( 1 - \frac{( sample - lowcontrol )}{( {highcontro}^{′} l - lowcontrol )} )$$

### Results

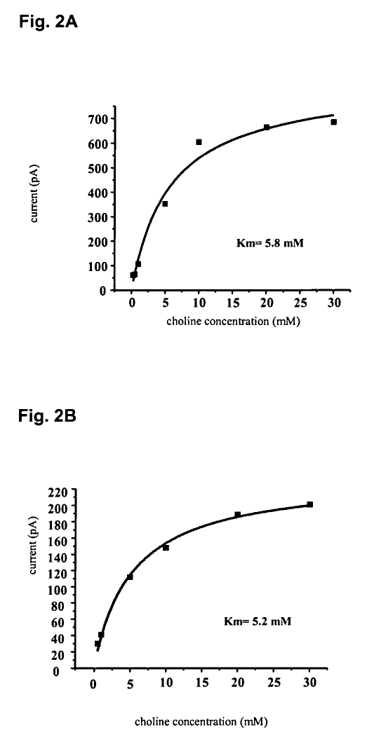

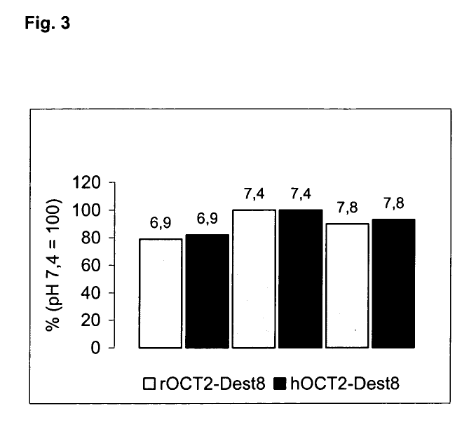

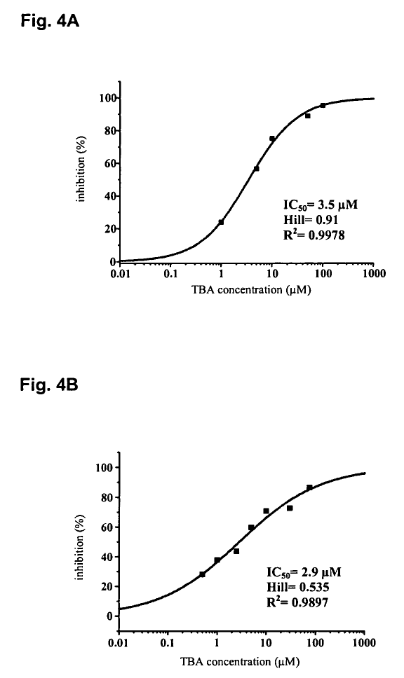

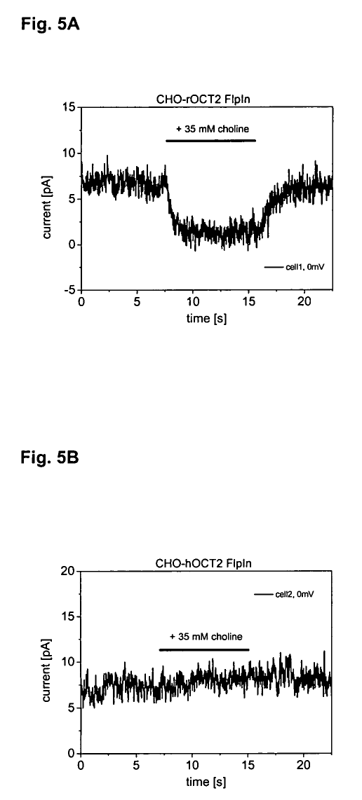

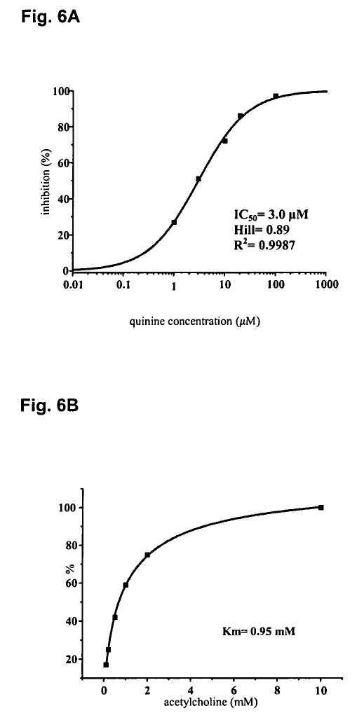

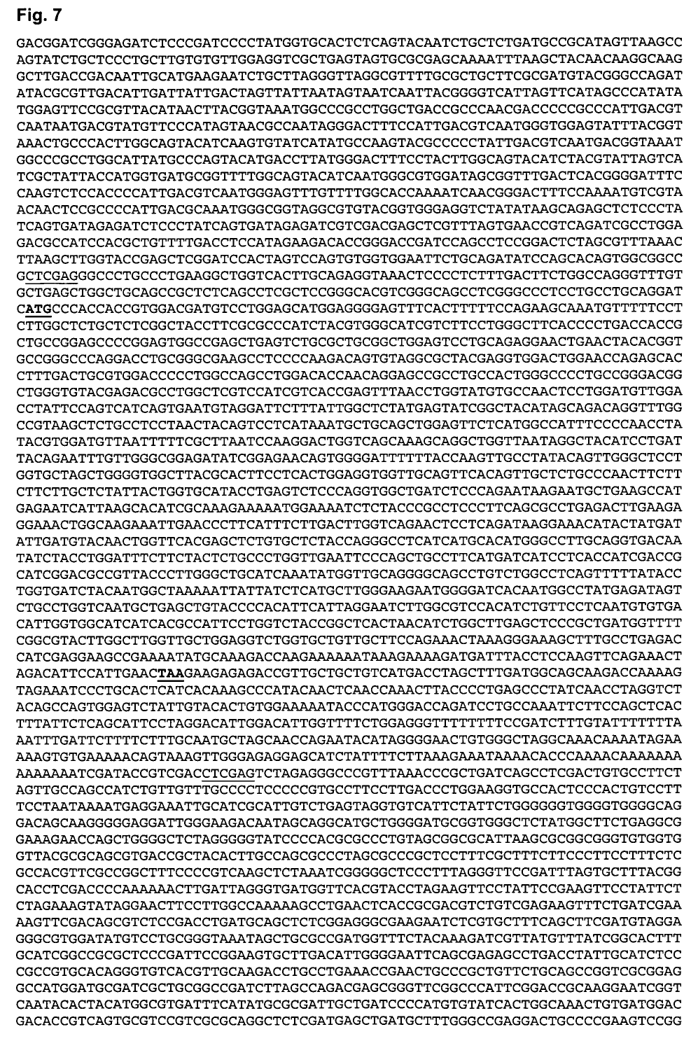

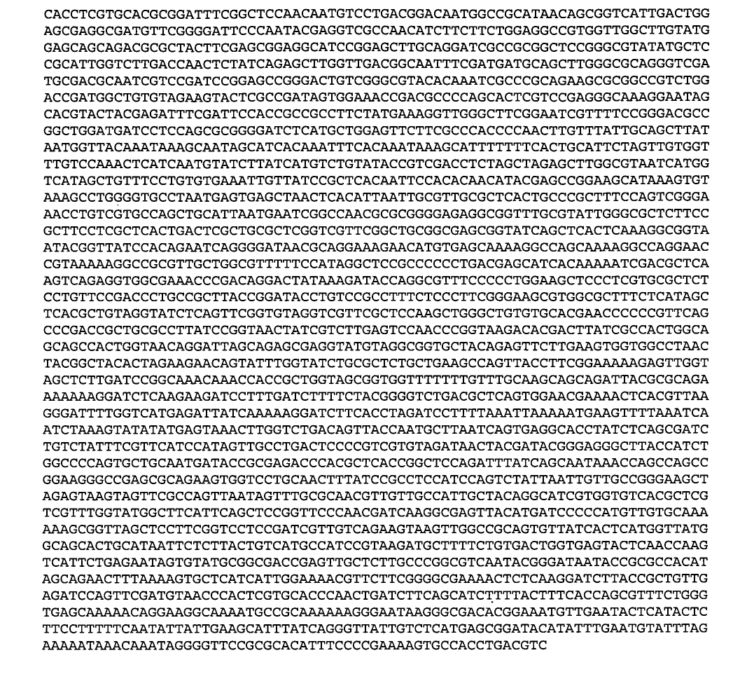

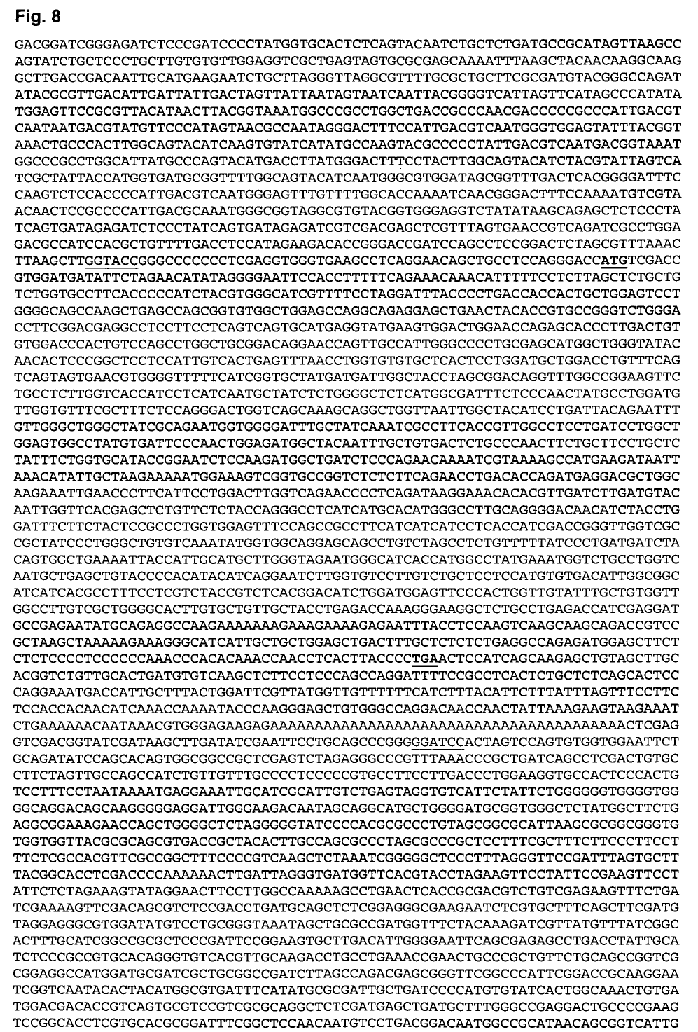

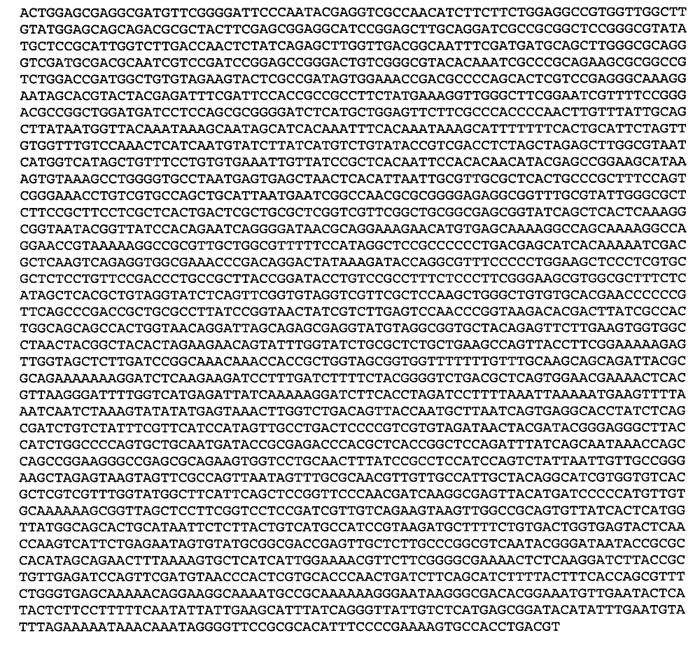

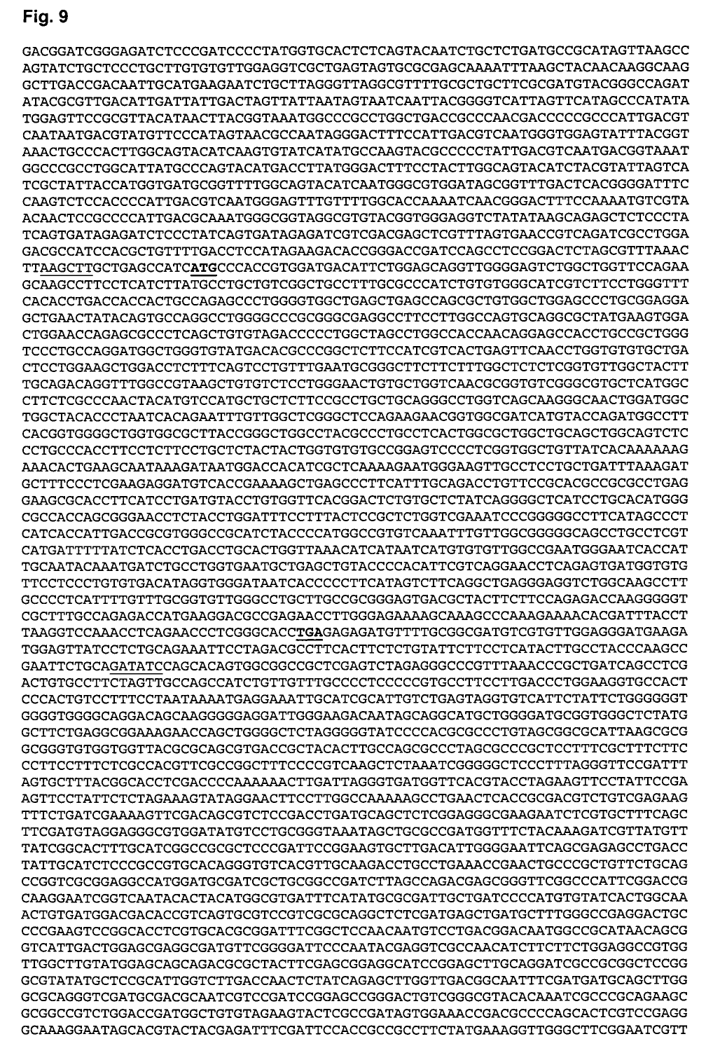

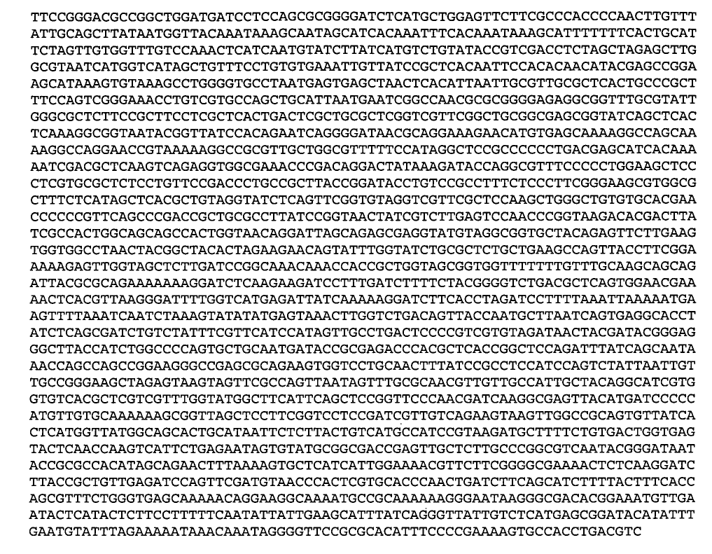

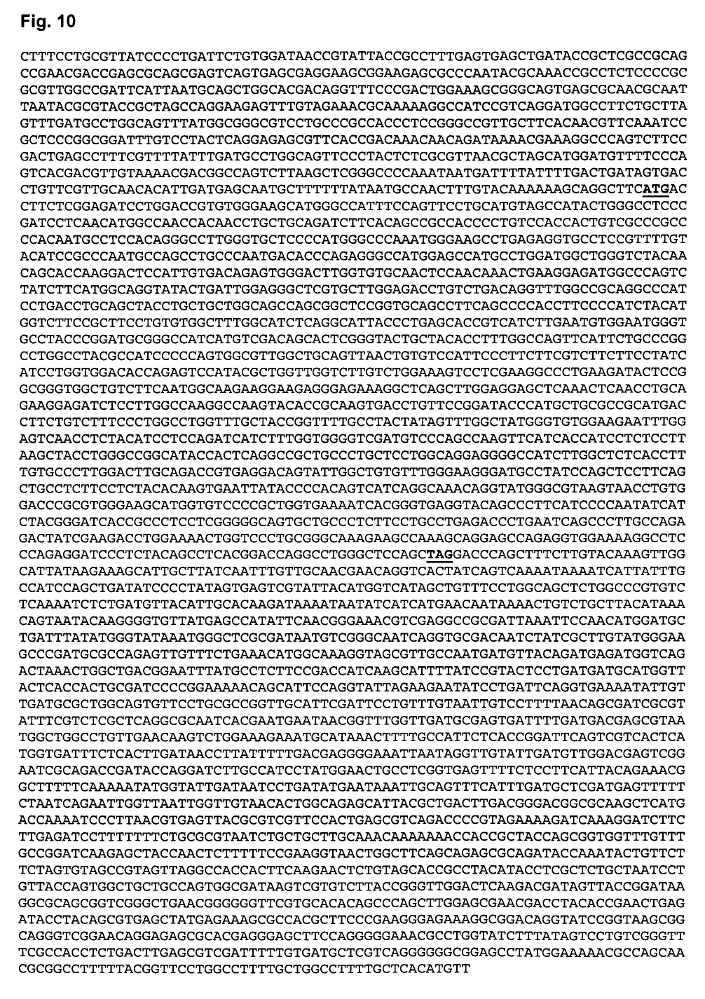

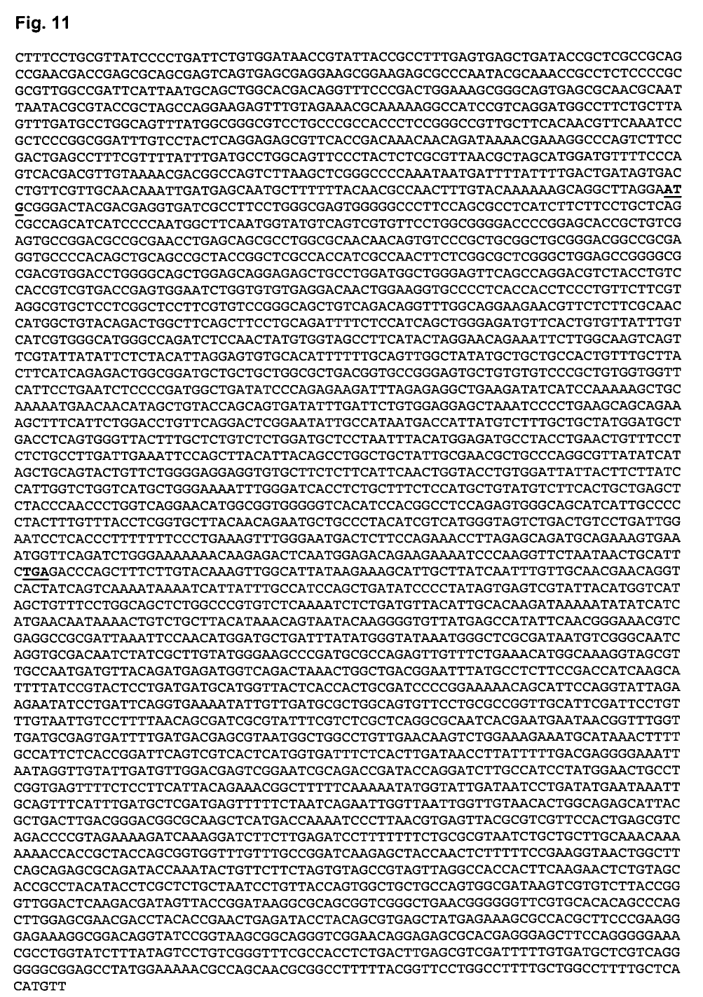

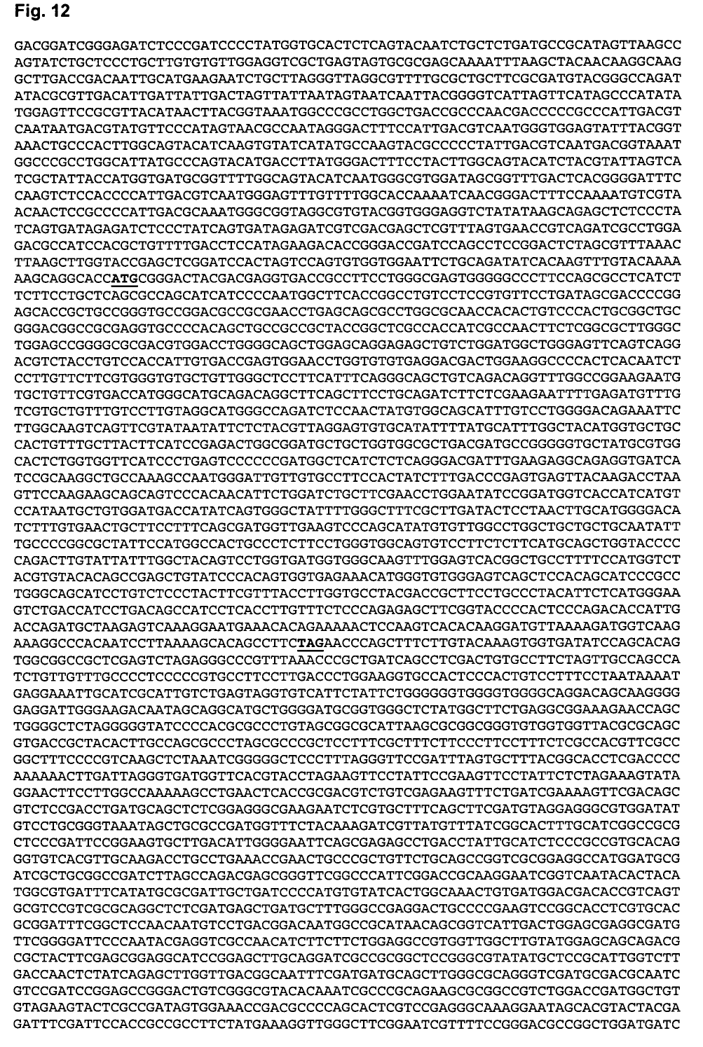

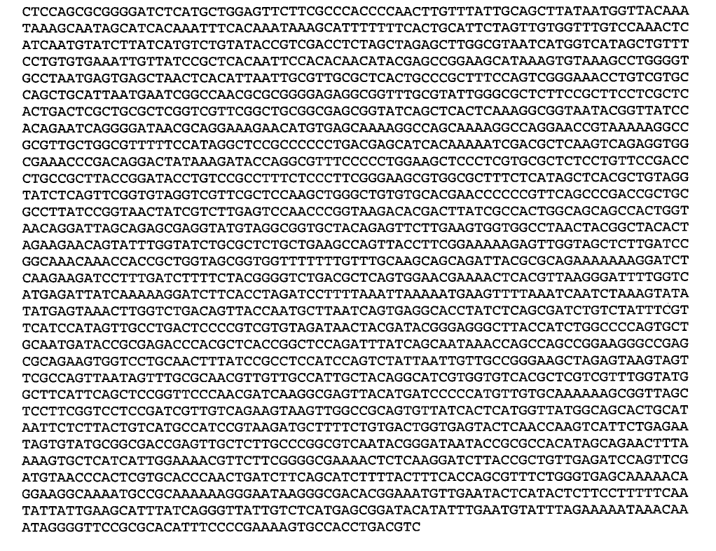

1. FIGS. 1A and 1B show electrical responses upon addition of choline containing activating solution to sensors with immobilized membranes harbouring rOCT2 and hOCT2 respectively before (black trace) and after inhibition (grey trace). The peak amplitude is equivalent to the initial activity of the transporters; the decay has to be attributed to the charging of the capacitance of the sandwich structure of the biosensor.
2. FIGS. 2A and 2B show the influence of the choline-concentration on the amplitude of the electrical response (high control) on rOCT2 and hOCT2 containing membranes respectively.
   According to the results of a choline-concentration titration a choline-concentration of 100 mM was used in the following tests as this allowed to measure signals with high amplitude.
3. Measured pH dependence showed highest protein activity at pH 7.4, which therefore was used in subsequent tests (FIG. 3). For inhibition experiments the choline concentration was decreased to 10 mM (in the range of KM-value for detecting competitive inhibitor effects). The IC50 for a standard inhibitor of the OCT (TBA) was determined to 3.5 μM for rOCT2 (FIG. 4A) and 2.9 μM for hOCT2 (FIG. 4B) respectively.
4. By using the parameters defined above different membrane preparations from recombinant cell lines were compared. Best results were obtained with a CHO cell line. Insect cell preparations yielded high quality signals, however with a rundown not suited for IC₅₀ determination.
5. The CHO cell line was further monitored via manual patch clamp electrophysiology considered as gold standard for ion transporter research. For rOCT2 electrical currents were hardly for hOCT2 not detectable and IC50 values could not be determined (FIGS. 5A and 5B).
6. For further evaluation of the sensitivity of the signal further substrates and inhibitors were tested. FIGS. 6A and 6B show these examples.

Along with the assays reported here for the OCT2s further family members, e.g. hOCT1 or hOCT3, and constructs were cloned and generated. Cell lines were generated utilizing Invitrogen's Flpln- and T-REX System (Cat. No. R758-07).

1. A method for determining the activity of the organic cation transporter (OCT), said method comprising the consecutive steps of:
(a) providing a cell free electrophysiological sensor chip containing a solid-supported sensor electrode and a lipid layer containing the OCT located in the immediate spatial vicinity to the sensor electrode, whereas the sensor electrode is electrically insulated relative to the solutions used and to the lipid layer,
(b) treating the sensor chip with an ion-containing non-activating solution, treating the sensor chip with an ion- and substrate containing activating solution, and measuring an electric signal.

2. The method of claim 1, wherein the OCT is selected from the group consisting of OCT1 (SLC22A1), OCT2 (SLC22A2), OCT3 (SLC22A3), OCTN1 (SLC22A4), and OCTN2 (SLC22A5).

3. The method of claim 1, wherein the OCT is of mammalian origin, particularly from rat, mouse, rabbit, pig, guinea pig, drosophila melanogaster, caenorhabditis elegans or human, more particularly human OCT1 (SLC22A1).

4. The method of claim 1, wherein the sensor electrode comprises a metallic material or an electrically conductive metal oxide.

5. The method of claim 1, wherein the solid-supported sensor electrode is a glass- or a polymer-supported sensor electrode.

6. The method of claim 1, wherein the lipid layer is attached to the sensor electrode via a chemical bond, particularly via his-tag coupling or streptavidin-biotin coupling, or via hydrophobic, hydrophilic or ionic forces.

7. The method of claim 1, wherein the sensor electrode is electrically insulated by at least one insulating monolayer.

8. The method of claim 1, wherein the sensor electrode is first washed with an ion-containing washing solution.

9. The method of claim 8, wherein the ion-containing solution contains univalent and bivalent ions selected from the group consisting of Na⁺, K⁺, Mg²⁺ and Ca²⁺.

10. The method of claim 8, wherein the total concentration of the ions in the ion-containing solutions is from about 100 mM to about 1000 mM.

11. The method of claim 9, wherein the concentration of the univalent ions in the ion-containing solutions is from about 300 mM to about 400 mM.

12. The method of claim 9, wherein the concentration of the bivalent ions in the ion-containing solutions is from about 2 mM to about 10 mM, particularly from about 5 mM to about 8 mM, more particularly about 5 mM.

13. The method of claim 8, wherein the ion-containing solutions further contain a buffer.

14. The method of claim 1, wherein the substrate of the activating solution comprises an organic cation, a cationic xenobiotic, a cationic vitamin, a combination of an organic cation, a cationic xenobiotic, or a cationic vitamin, a primary amine, a secondary amine, a tertiary amine, a quaternary amine, a biogenic amine, a lipophilic compound, a steroid or an organic anion.

15. The method of claim 1, wherein the electric signal is measured using an amperometric means, a potentiometric means, or a combination of an amperometric means and a potentiometric means.

16. The method of claim 1, wherein step (b) is carried out at least 2 times.

17. The method of claim 1, wherein the method is carried out in the presence of a chemical compound.

18. A method for determining whether a chemical compound modulates the activity of an organic cation transporter, comprising the steps of:
(a) determining the activity of the organic cation transporter (OCT) using the method of claim 1 absent the chemical compound,
(b) determining the activity of the (OCT) using the method of claim 1 absent the chemical compound in the presence of the chemical compound, and
(c) determining whether there is a difference in the activity of the OCT measured in step (a) and step (b),
wherein a difference in the activity of the OCT measured in step (a) and step (b) is indicative that the chemical compound modulates the activity of the OCT.

19. The method of claim 18, wherein the method is carried out in the presence and/or in the absence of the substrate of the activating solution.

20. A method for identifying a chemical compound that modulates the activity of OCT, said method comprising the consecutive steps of:
(a) carrying out the method of claim 1, and
(b) identifying the chemical compound.

21. The method of claim 20, wherein the chemical compound is an organic cation, a cationic xenobiotic, a cationic vitamin, a combination of an organic cation, a cationic xenobiotic or a cationic vitamin a biogenic amine, a primary amine, a secondary amine, a tertiary amine, a quaternary amine, a lipohilic compound, or an organic anion.

22. The method of claim 20, wherein the chemical compound is an inhibitor of OCT.

23. The method of claim 17, wherein the chemical compound is present in a chemical compound library.

24. A cell free electrophysiological sensor chip of claim 1.

25. The sensor chip according to claim 24, further comprising a data acquisition device for acquiring measurement data from the electrode, and optionally an exchange means, mixing means or a combination of an exchange means and a mixing means for making available exchanging, mixing, or exchanging and mixing the ion-containing solutions.

26. The sensor chip of claim 24 in the form of a microplate or microtiter plate.

27. An apparatus containing the sensor chip of claim 24, a reference electrode, a data acquisition device for acquiring measurement data from the electrode, and an exchange means, a mixing means, or a combination of an exchange means and a mixing means for making available, exchanging and/or mixing the ion-containing solutions, a flow analysis device, a power supply, a computer and an autosampler.

28. The apparatus of claim 27, wherein the reference electrode is a Pt/Pt, Ag/AgCl or indium tin oxide electrode.

29. A kit containing
(a) a cell free electrophysiological sensor chip of claim 24,
(b) at least one ion-containing washing solution, and optionally
(c) a substrate comprising an organic cation, a cationic xenobiotic, a cationic vitamin. a combination of an organic cation, a cationic xenobiotic, or a cationic vitamin, a primary amine, a secondary amine, a tertiary amine, a quaternary amine, a biogenic amine, like epinephrine, norpeinephrine or carnitine or a lipophilic compound, compounds like quinine, quinidine or a steroid steroids like corticosterone or an organic anion.

30. The method of claim 4, wherein the electrically conductive metal oxide comprises gold, platinum, silver or indium tin oxide.

31. The method of claim 5, wherein the glass- or a polymer-supported sensor electrode comprises borofloat-glass-supported sensor electrode or a borofloat-glass-supported gold electrode.

32. The method of claim 6, wherein the chemical bond that attaches the lipid layer to the sensor electrode is a his-tag coupling, streptavidin-biotin coupling, hydrophobic forces, hydrophilic forces, or ionic forces.

33. The method of claim 7, wherein the insulating monolayer comprises at least one insulating amphiphilic organic compound, at least one insulating membrane monolayer, or a mercaptan layer as an under layer facing the sensor electrode and a membrane monolayer as an upper layer facing away from the electrode.

34. The method of claim 33, wherein the mercaptan layer comprises octadecyl thiol.

35. The method of claim 10, wherein the total concentration of the ions in the ion-containing solutions is from about 200 mM to about 500 mM, more particularly from about 300 mM to about 500 mM, most particularly about 435 mM.

36. The method of claim 35, wherein the total concentration of the ions in the ion-containing solutions is from about 300 mM to about 500 mM.

37. The method of claim 10, wherein the total concentration of the ions in the ion-containing solutions about 435 mM.

38. The method of claim 9, wherein the concentration of the bivalent ions in the ion-containing solutions is from about 5 mM to about 8 mM.

39. The method of claim 38, wherein the concentration of the bivalent ions in the ion-containing solution is about 5 mM.

40. The method of claim 13, wherein the buffer is a HEPES/NMG, 30±10 mM, pH 7.0±1.0 buffer.

41. The method of claim 14, wherein the quaternary amine is selected from the group consisting of choline, acetylcholine, nicotine, N1-methylnicotineamide, morphine, 1-methyl-4-phenylpyridinium, procainamide, tetraethylammonium, and tributylmethylammonium, the biogenic amine is selected from the group consisting of epinephrine, norpeinephrine and carnitine, the lipophilic compound is selected from the group consisting of quinine and quinidine, the steroid is corticosterone and the organic anion is selected from the group consisting of para-amino hippuric acid and probenecid.

42. The method of claim 16, wherein step (b) is carried out 2 to 4 times.

43. The method of claim 17, wherein the chemical compound is an inhibitor of OCT.

44. The sensor chip of claim 25 in the form of a microplate or microtiter plate.
